(19) United States
(12) Patent Application Publication
(10) Pub. No.: US 2002/0014017 A1
(43) Pub. Date: Feb. 7, 2002

(54) System using anthropometric frame for measurement of bony spatial relationships

(76) Inventor: David A. Egan, Dartmouth (CA)

Correspondence Address: Simpson, Simpson & Snyder,, 5555 Main Street,, Williamsville, NY, 14221, US

(21) Appl. No.: 09/839,772

(22) Filed: Apr. 20, 2001

(30) Foreign Application Priority Data
Apr. 28, 2000 (CA) 2,307,063

(51) Int. Cl. A61B 5/103

(52) U.S. Cl. 33/512

(57) ABSTRACT

Disclosed is an anthropometric frame system comprising a base, a foot frame device for selectively measuring or placing feet of a subject in a preferred or selected stance orientation on the base and an orthogonal frame device for measuring the spacial relationships of various bony points on the subject's body. The orthogonal frame device comprises means mounted to the base for horizontally supporting a crossbar in selected vertical positions, the crossbar having means for supporting a first measurement device for lateral measurements. The crossbar also includes means for supporting a second measurement device for vertical measurements and the means for supporting the second measurement device also supporting a third measurement device for front to back measurements whereby orthogonal spacial relationship between bony points on a subject's body can be measured. The foot frame device is selectively positioned on the base relative to the orthogonal frame device whereby orthogonal measurements are taken when the subject is in the preferred or selected stance orientation in the foot frame. Also disclosed is a method of measuring the spacial orthogonal relationships between selected bony points on a subject using the an anthropometric orthogonal frame system.

This application claims the benefit of the filing date under 35 U.S.C. 119(a) of Canadian Patent Application No. 2,307,063 filed on Apr. 28, 2000.

### FIELD OF THE INVENTION

The invention relates to a system using an anthropometric frame which allows measurements of the spatial relationship between bony points on animals such as the measurement of bony pelvic landmarks and the stance position of humans.

### BACKGROUND OF THE INVENTION

Skeletal asymmetry has been frequently associated with altered function and is frequently thought to be a contributing factor in a variety of pain syndromes attributed to asymmetry within the musculoskeletal system, for example, low back pain in humans. Many of these musculoskeletal problems account for enormous costs both directly—time off work and compensation for the sufferer—and indirectly—lost productivity. To a very great extent, current practice relies on the use of subjective measurements for decisions on interventions and care and objective outcome measures are scarce or lacking. It is of particular interest that this widespread subjective approach is particularly noticeable in the literature describing therapeutic interventions for relatively minor asymmetries. It is abundantly clear that objectivity of measurement, therapeutic planning and measurement of outcome are lacking.

Subjective examination of skeletal asymmetry in human subjects is frequently performed by visual-palpatory assessment of the whole body and selected parts. Although tape measures and goniometers are frequently employed in an attempt to provide more objectivity, these techniques have been shown to lack reliability and validity. The usual gold standard for the measurement of skeletal asymmetry entails the use of ionizing radiation, for example radiology, however, these procedures are expensive, time consuming and potentially hazardous to health. It is also important to recognize that measurements of pelvic asymmetry taken by most clinicians/researchers typically are restricted to front (anterior), or back (posterior), or front to back (antero-posterior or pelvic tilt) which do not give an accurate 3-dimensional description.

Accordingly, a system and a related frame for the accurate measurement of bony spatial relationships is needed. It will be of value for health practitioners such as orthopaedic surgeons, physiotherapists, chiropractors, osteopaths who diagnose and treat asymmetries and for technicians involved in the design and construction of lower limb prostheses and orthoses.

### SUMMARY OF THE INVENTION

The system of the invention uses a frame having stability and rigidity sufficient that accuracy of measurement, combined with minimum wear and tear, are maintained.

The invention in one broad aspect pertains to an anthropometric frame system comprising a base, a foot frame device for selectively measuring or placing feet of a subject in a preferred or selected stance orientation on the base and an orthogonal frame device for measuring the spacial relationships of various bony points on the subject's body. The orthogonal frame device comprises means mounted to the base for horizontally supporting a crossbar in selected vertical positions, the crossbar having means for supporting a first measurement device for lateral measurements. The crossbar also includes means for supporting a second measurement device for vertical measurements and the means for supporting the second measurement device also supporting a third measurement device for front to back measurements whereby orthogonal spacial relationship between bony points on a subject's body can be measured. The foot frame device is selectively positioned on the base relative to the orthogonal frame device whereby orthogonal measurements are taken when the subject is in the preferred or selected stance orientation in the foot frame.

The invention in another aspect pertains to a method of measuring the spacial orthogonal relationships between selected bony points on a subject, comprising the steps of (a) providing an anthropometric orthogonal frame system comprising a base and a foot frame device and an orthogonal frame device selectively positioned on the base, the orthogonal frame device comprising means mounted to the base for horizontally supporting a crossbar in selected vertical positions, the crossbar having means for supporting a first measurement device for lateral measurements and the crossbar including means for supporting a second measurement device for vertical measurements and the means for supporting second measurement device also supporting a third measurement device for front to back measurements, the third measurement device including a pointer and the foot frame device configured to permit placing of feet of the subject in a preferred or selected stance orientation on the base, (b) locating the feet of the subject on the base in association with the foot frame device, (c) locating the crossbar at a selected height relative to the area of the subject to be measured, (d) locating the pointer associated with the third measurement device at a desired location on the subject and recording measurements from at least two of the first, second and third measurement devices, (e) locating the pointer associated with the third measurement device at a second desired location on the subject who remains stationary in the same position as when the first measurements were recorded (f) recording the second measurement and (g) determining the spacial relationship between the first and second locations from the measurements recorded.

The system preferably includes a frame system having two components or devices with an optional component to the system.

The first component, device or part of the system is an adjustable foot frame device on a base which can be used to measure the subjects preferred stance orientation or which may be used to place the subject in a predetermined stance position facing forwardly or backwardly.

The second component, device or part of the system is an orthogonal frame device comprised of a 3-dimensional measurement component, (3-D component), which is mounted on two vertical poles which readily permit the 3-D component to be adjusted to different levels on the poles specifically so that the system can accommodate individuals from varying height groups. This part of the system determines the dimensions of the control volume. The measurement scales are preferably metric and all adjustable parts have locking screws.

The optional component or part of the system is preferred for the measurement process. A simple adjustable support such as a walking frame, (like those readily found in most clinics and nursing homes), is used to control the subject's postural sway and provide stability during the measurement process. However, other forms of like supports are possible.

More particularly, the system is preferably mounted on a solid base having an upper surface which can be easily cleaned and has a grid marked thereon. The base is equipped preferably with levelling screws or like means so that it can be levelled if the floor or other surface on which it is located is not level. The base can include visual aids for levelling the base or such aids can be separate.

The foot frame mounted on the base permits measurement of a subject's preferred foot position or to set a prescribed foot position, that is, the stance angle and distance between feet. The foot frame is comprised of a horizontal bar and two outer bars adjacent the base, the horizontal bar being moveable forward and back relative to the base and grid thereof

The two outer or lateral bars are pivotally and slidably connected at one end of the outer bars to the horizontal bar.

Although protractors can be attached where each outer bar is pivotally connected to the horizontal bar to measure a stance angle, the use of separate protractors is an alternative.

The 3-D component comprises two laterally spaced, vertical poles located towards the front of the base and are marked with identical measurement scales. A horizontal bar is vertically adjustably mounted to the two poles. Three precision orthogonal measuring devices are adjustably mounted to the horizontal bar. The precision measuring devices are used to measure differences in front to back, up and down and side to side distances.

The optional support frame is height adjustable and designed for stabilizing the subject and restricting postural sway during testing and measurement. Such a device can be incorporated with the base but a single adjustable walker frame is usable and therefore the support frame is optional as a component necessarily connected to the base.

The system is designed to be consistently accurate, robust and stable, relatively inexpensive, non-invasive (ionizing radiation free) and with reasonable portability within a clinical facility. It is easy to use and provides the clinician or researcher with immediate and accurate data for diagnostic and follow-up assessments. Calculations of the ratios which define different pelvic skeletal asymmetries can be very quickly obtained, require very simple mathematics and are readily derived from anterior and posterior measurements alone.

The purpose of the system, including the components, is to provide objective and accurate measurements of the spatial relationships between bony points on human subjects. Although it has been designed specifically for the measurement of bony pelvic landmarks and stance position, the frame could be adapted for other human body measurements. There is a distinct advantage of having a relatively small control volume in which highly accurate measurements can be taken, especially when the control volume can be moved into different positions to accommodate for individuals of widely differing height and for selected body measurements.

The frame was tested for accuracy and found to be accurate to 1.0 mm within a typical measurement volume, or control volume, of approximately 40 cm width×20 cm height×15 cm depth. A control volume establishes the outermost boundaries for accurate measurements. Adhesive markers are placed on the subject for identification of the bony points and for the measurements. This method has also been statistically tested in the lab and shown to be highly reliable for the accuracy of repeated marker placements and measurement. The obtained raw measurements are used to calculate asymmetry ratios, thus providing normalized data which can be used to make objective comparisons between subjects.

### BRIEF DESCRIPTON OF THE DRAWINGS

FIG. 1 is a front objective view of the frame of the invention with a foot component and a 3-dimensional measurement component, (3-D component), of the system, both associated with a base support.

FIG. 2 is a partial sectional view taken along line 2-2 of FIG. 1 showing the lock screw and slot connection of the frame of foot component with the base.

FIG. 3 is a partial sectional view taken along line 3-3 of FIG. 1 showing the lock screw slot connection of the lateral or outer bars of the foot frame with the main bar of the foot frame.

FIG. 4 is a sectional view of the connection between the bar of the 3-D component and a vertical post taken along line 4-4 of FIG. 1.

FIG. 5 is an enlarged view of a portion of the 3-D component as seen in FIG. 1.

FIG. 6 is a left side view of the 3-D component.

FIG. 7 is a sectional view of the connection of the pointer ruler taken along line 7-7 of FIG. 6.

FIG. 8 is a top view of the foot frame showing a subject's stance position.

FIG. 9 is an oblique view of the measurements of markers on the back of a subject for posterior analysis.

### DETAILED DESCRIPTION OF THE PREFERRED EMBODIMENT

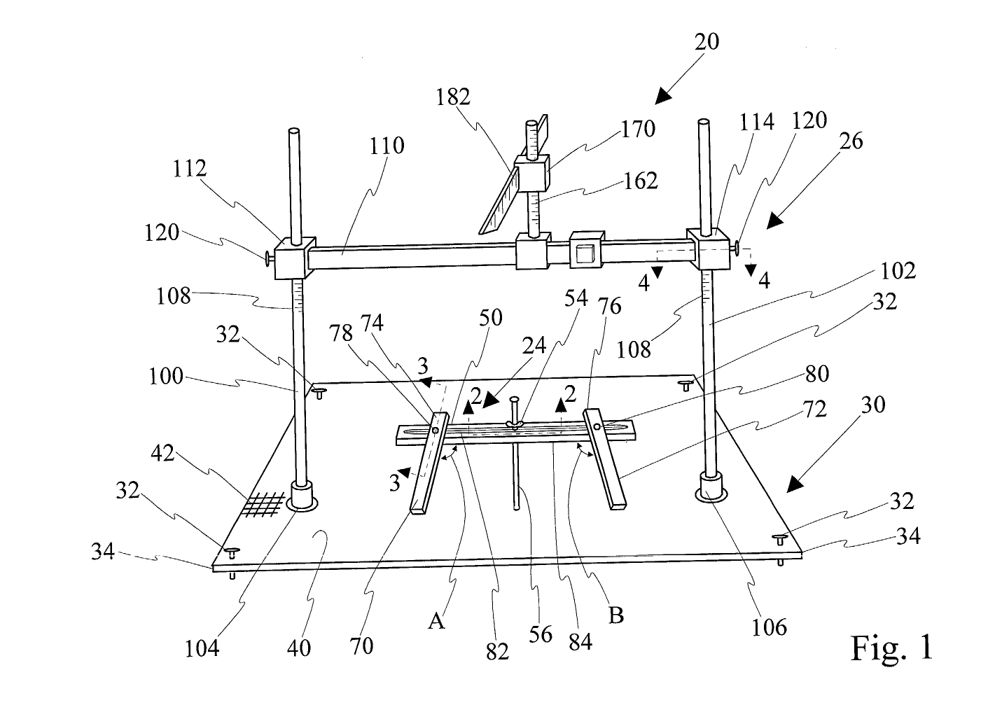

Referring to FIG. 1, the inventive anthropometric frame 20 is shown with foot or stance component 24 and 3-dimensional component, (3-D component), 26 on base 30.

Base 30 is a sheet of material such as wood or plastic with levelling screws 32 at each corner 34 whereby base 30 can be levelled on a support surface such as the floor 38. Base 30 has an upper surface 40 covered with material capable of being easily cleaned and on which a grid can be formed. Surface 40 preferably is a laminex surface with a matrix grid 42 of preferably one centimeter squares marked thereon, only a small portion of grid 42 being shown.

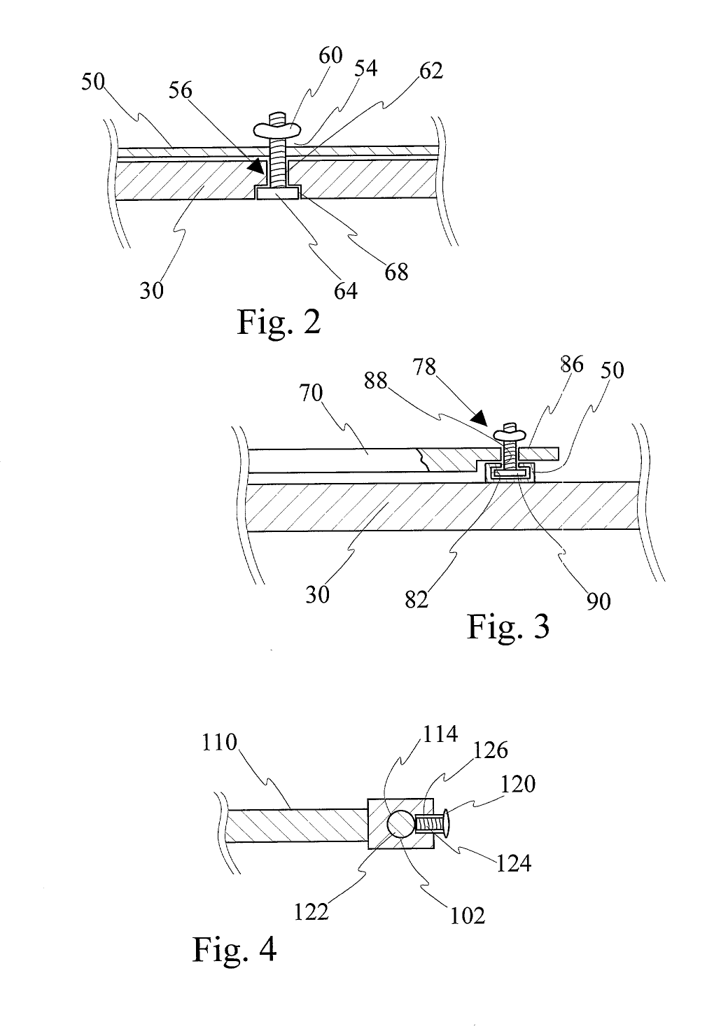

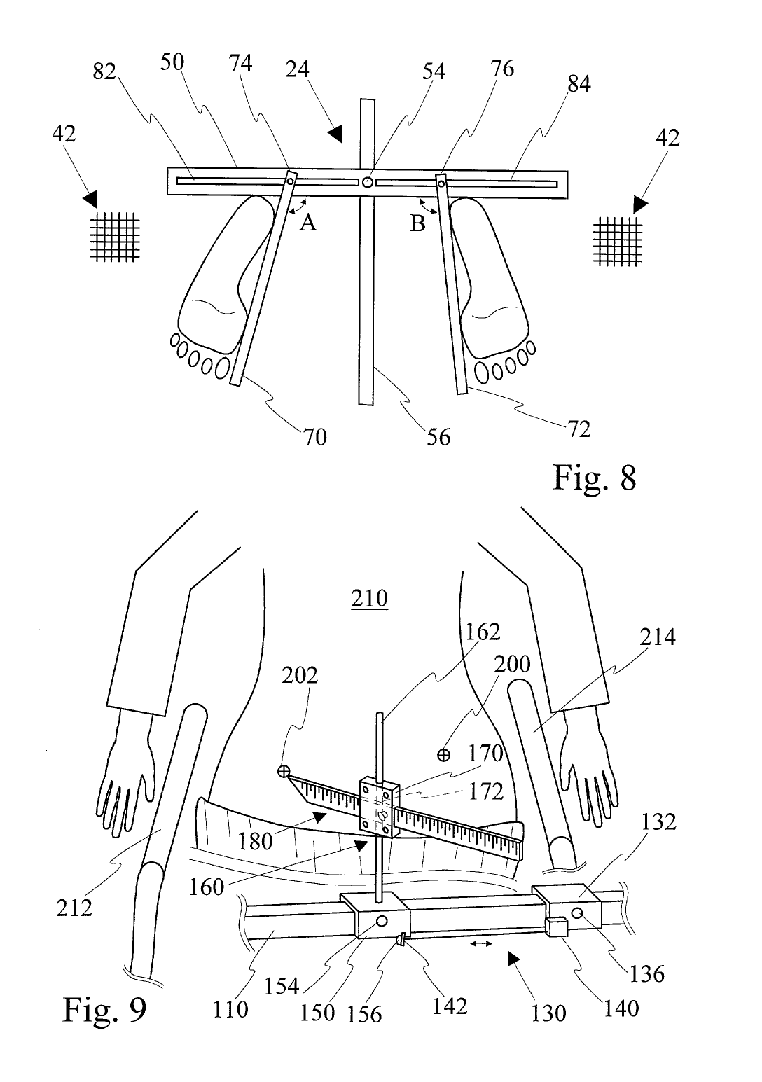

Turning more particularly to FIGS. 1, 2, 3 and 8, foot or stance measurement component 24 comprises a bar 50 which is slidingly mounted to base 30 through a locking screw 54 and associated slot 56 in base 30. Bar 50 is selectively moveable in a forward or rearward movement and can be detachably secured in a selected position by the lock screw 54 in cooperation with slot 56 as illustrated in sectional view in FIGS. 2 and 8.

Lock screw 54 is typical of the other lock screws referred to herein including wing nut 60, threaded shank 62 and circular head 64 adapted to run in elongate recess 68 of the slot 56 as shown in FIG. 2.

Laterally spaced bars 70, 72 are pivotally connected adjacent ends 74, 76 respectively to bar 50 and can be moved laterally inwardly or outwardly along respective portions of bar 50 through a lock screw 78, 80 and slots 82, 84 respectively. FIG. 3 is a partial sectional view along lines 3-3 of FIG. 2 showing the connection of bar 70 with bar 50, the connection of bar 72 being similar to that of bar 70. Winged screw 78 includes shank 56 extending through bore 88 in bar 70 and slidably received in slot 82 of bar 50, with head 90 running in elongate recess 92 in bar 50.

The purpose of the foot component 24, (as seen in FIG. 8), is to permit measurement of a subject's preferred foot position or to set a prescribed foot position (stance angle and distance between feet). The foot component 24 is comprised of the horizontal bar 50 and the two outer bars 70, 72. The horizontal bar being adjustable forwardly and rearwardly in slot 56 in the center of the base 30. Bar 50 can be set in line with the marker grid 42 on the base 30 or at any selected angle. Each outer bar 70, 72 may be adjusted towards or away from the center of the horizontal bar, in respective slots 82, 84 in horizontal bar 50. Protractors may be attached where each outer bar is pivotally mounted to the horizontal bar to measure the stance angle denoted A and B in FIGS. 1 and 8. Alternatively, protractors may be used separately. It should be noted that the foot frame device 24 may be rotated 180 about pivot 54 so that the stance orientation may be set the same for both anterior and posterior measurements with the orthogonal frame device.

Turning particularly to FIGS. 1, 4-7 and 9, the 3-D frame 26 comprises laterally spaced rigid poles 100, 102 vertically mounted to base 30 through collars 104, 106 respectively, poles 100, 102 being mounted perpendicular to and towards the front of base 30, (collars 104, 106 are in the form of threaded pipe sockets or plain sockets with locking screws). Poles 100, 102 are marked with identical measurement scales 108, only parts of which are shown. It will be appreciated that poles 100, 102 can be detachably mounted to base 30 but in any event, poles 100, 102 are precision mounted so that the vertical measurements scale 106 on each pole will be in lateral (horizontal) alignment.

Rigid horizontal bar 110 is mounted for selected vertical movement relative to vertical poles 100, 102 through blocks 112, 114 and a combination lock wing nut screw 120 and block bore 122 as shown further in sectional view in FIG. 4, screw 120 having shank 124 threaded in bore 126 of block 114.

It will be apparent however that any other mechanism to mount bar 110 horizontal to poles 100, 102 so that bar 110 is movably secured to poles 100, 102 is appropriate, including poles with a slot therein.

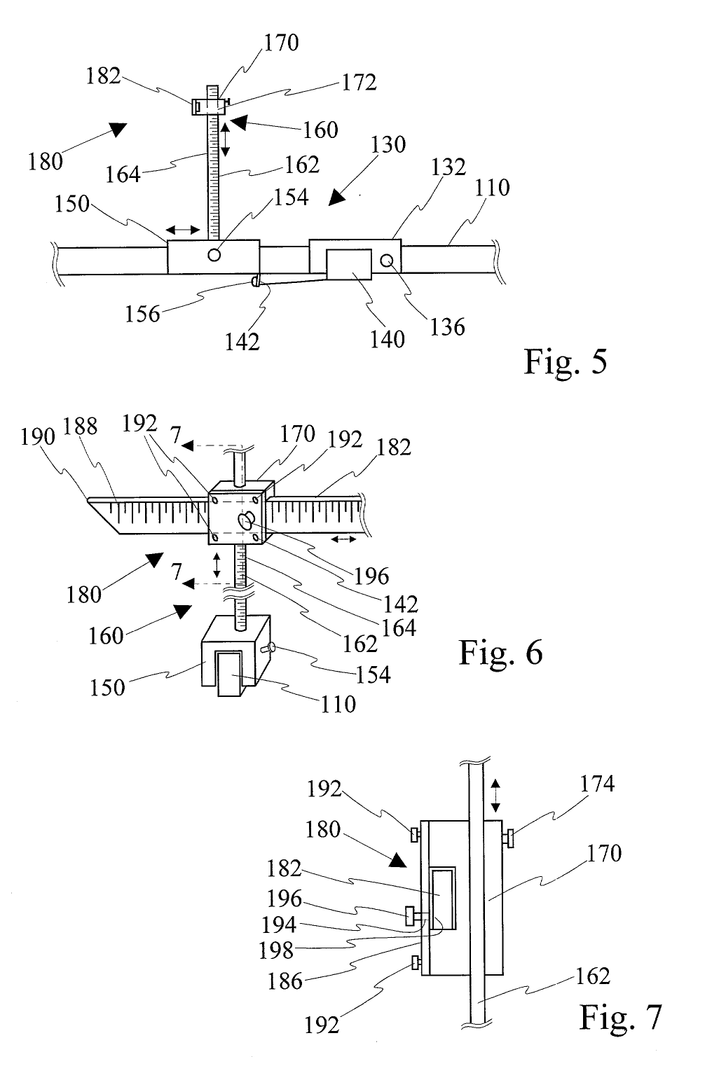

A first measurement device 130, more particularly shown in FIG. 5, includes a horizontally movable first sliding block 132 having a U-shaped cross-section for riding on bar 110 with a lock screw 136 capable of securing block 132 at a selected location on bar 110. A spring loaded tape measure 140 of usual construction with outer tape end 142 is securely mounted on block 132. Further, a second sliding block 150, also U-shaped in cross-section, (see FIGS. 5 and 9), is slidably mounted on bar 110, second sliding block 150 having lock screw 154 whereby block 150 may be positively secured in a selected position on bar 110. End 142 of tape measure 140 is detachably fastened in catch 156 of block 150 whereby movement of sliding block 150 on bar 110 relative to block 132 causes tape measure 140 to extend or retract depending on the direction of relative movement between block 150 and block 132.

Turning to FIGS. 6 and 7, a second measurement device 160 comprises a vertically scaled pole 162 fixed to and extending vertically upwardly from sliding block 150, pole 162 having metric scale 164 thereon, only part of which is shown.

Further, block 170, having throughbore 172, is mounted on pole 162 for selective sliding movement along pole 162, with lock screw 174 selectively securing block 170 at desired positions on pole 162.

A third measurement device 180 comprises pointed horizontally disposed ruler 182 being selectively secured in slot 184 in block 170 in a desired position by a clear plastic cover 186 secured by four locking screws 192. Threaded lock screw 194 has head 196 and its shank end 198 can be pressed against ruler 182 to hold it in a selected position within slot 184. Alternatively a tight sliding fit between slot 184 and ruler 192 is possible. Ruler 182 has ruler or scale 188 thereon (partly shown) and pointed outer end 190.

Accordingly, the 3-D component 20 provides three orthogonally mounted and adjustable measuring devices attached to horizontal bar 110, namely a first device 130 to measure the differences side to side, a second device 160 to measure the differences up and down and a third device 180 to measure differences from front to back.

Further particulars with respect to the measurement, (anterior measurements) and use of the 3-D component of the frame and system follow.

Assume that a subject has two anterior markers, one each on the left and right side of the pelvis and the person is standing on the base board facing towards the front of the system and the tester, i.e. looking forwardly from FIG. 1 and as the feet are shown in FIG. 8.

Step 1. The stance/foot position is measured or set to a prescribed position with frame device 24. (See FIG. 8).

Step 2. A walker frame (or similar support, the third component) is brought up behind the subject and the individual is instructed to steady themselves by holding onto the walker frame without taking weight through the arms.

Step 3. The horizontal rigid bar 110 is placed about level with the hip joint, checked for level and the locking screws 120 tightened.

Step 4. Lock screw 194 on clear plastic cover 186, lock screw 174 on block 170, lock screw 136 on the first sliding block 132 and the lock screw 154 on the second sliding block 150 respectively are slackened.

Step 5. The end 190 of horizontal pointer rule 182 is aligned with the bony point marker on the left side of the subject (testers right), as looking at FIG. 1, ensuring that both first and second sliding blocks 132, 150 are together (in this position the spring loaded tape measure 140 will be at zero). Lock screws 136, 154, 174 and 194 are then tightened.

Step 6. The height of the subject's left marker and distance from the frame are recorded from scales 162 and 188. These positions are used as the reference point for subsequent measurements.

Step 7. The pointer 190 is then retracted from the subject by loosening cover lock screw 194 and lock screws 154 and 174, (but not lock screw 136 of first sliding block 132). The second sliding block 150 (on which the vertical pole 162 and horizontal ruler 182 are mounted) is then moved to align with the subject's right side marker, (left side of tester) and the locking screws 154, 174 and 194 are tightened. The first block 132 remains in situ. The height of the subject's right side marker and distance from the frame are then recorded and the width apart of the markers can be simultaneously recorded because the spring loaded tape measure 140 will have been extended by the exact same amount.

Step 8. Using the asymmetry ratio method, the height difference divided by the width separation of the markers will give the anterior asymmetry ratio which is the gradient (slope) or tangent of the angle to the horizontal between the two markers.

As well, the actual measurements from the floor to each marker can be recorded from scale 106 combined with scale 164.

Step 9. A similar procedure is repeated for the posterior measurements with posterior markers 200, 202 as shown in FIG. 9.

More particularly, lock screw 54 on foot frame device is loosened and the foot frame, with orientation of bars 70 and 72 fixed in the same orientation as shown in FIG. 8, is rotated 180. The subject's feet are then located within the foot frame device with the same stance and angular orientation as they were in during the anterior measurements. With the subject 210 now facing the back of the device as shown in FIG. 1, skin markers 200, 202, (FIG. 9), are applied to a bony projection on the back of each hip. Accordingly, relative front to back distances of the bony points are recorded from the pointed ruler 182, the point 190 of which is brought close to and aligned first with the skin marker 200. As ruler 182 can be moved up or down on vertical pole 162, the scale 164 on pole 162 is used to measure comparative heights when end 190 of ruler 182 is aligned with other bony points, such as the one identified by marker 202. With block 132 secured in the position where the first body marker 200 was recorded, block 150 can be slightly adjusted on bar 110 to permit ruler pointer 190 to be aligned with the opposite side body marker 202 and the distance between the blocks 132 and 150, which is exactly the same as the horizontal distance between the body markers 200, 202, can be recorded from the tape measure 140. The differences in height between the markers 200, 202 can also be measured using scale 164 on pole 162. FIG. 9 also shows the arms 212, 214 of a walker used by the subject 210 to steady him/herself as noted above in Step 2 for anterior marker measurements.

Thus, using asymmetry ratios, objective comparisons between the spatial relationships of the anterior, posterior, right and left halves of the selected bony points of the pelvis can be made and the spatial relationship between the stance position and pelvic asymmetry can be determined.

What we claim is:

1. An anthropometric frame system comprising:
a base;
a foot frame device for selectively measuring or placing feet of a subject in a preferred or selected stance orientation on said base;
an orthogonal frame device for measuring the spacial relationships of various bony points on said subject's body;
said orthogonal frame device comprising means mounted to said base for horizontally supporting a crossbar in selected vertical positions, said crossbar having means for supporting a first measurement device for lateral measurements, said crossbar including means for supporting a second measurement device for vertical measurements and said means for supporting said second measurement device also supporting a third measurement device for front to back measurements, whereby orthogonal spacial relationships between bony points on a subject's body can be measured; and
said foot frame device being selectively positioned on said base relative to said orthogonal frame device whereby orthogonal measurements are taken when said subject is in said preferred or selected stance orientation in said foot frame.

2. The frame system of claim 1 further wherein said foot frame device comprises a first member pivotally mounted and movably constrained for linear movement relative to said base and at least one second member is pivotally associated with said first member and adjustable relative to said first member whereby a foot of a subject placed on said base with a heel adjacent said first member and with the second member aligned with a side portion of the foot, angular orientation of the foot stance of the subject can be measured or set.

3. The frame system according to claim 2 wherein said foot frame device comprises two second members, each pivotably associated with said first member and spaced laterally whereby each one of said second members can be aligned with a side of a respective foot of said subject to measure the stance orientation or selectively locate the subject's feet in a preferred stance orientation.

4. The frame system according to claim 3 wherein said base includes a grid on its upper surface, at least in the area of said foot frame device.

5. The frame system according to claim 1 further comprising means for levelling said base.

6. The frame system of claim 1 wherein said means for horizontally supporting said crossbar comprises two laterally spaced poles vertically mounted to said base and each of said first and second vertical poles are marked with grid lines whereby said crossbar can be accurately located horizontally.

7. The frame system of claim 1 further including means selectively removable from said base whereby a subject can steady him/herself when standing on said base.

8. The frame system of claim 1 wherein said first measurement device includes two block members selectively slidable and lockable on said crossbar, one of said block members defining said means for supporting said second measurement device and means for measuring the distance between said two blocks on said crossbar whereby the lateral spacial relationship between said blocks can be measured.

9. The frame system of claim 1 wherein said second measurement device comprises a grid marked vertical pole and a block member selectively slidable along said vertical pole for supporting said third measurement device, said third measurement device comprising a pointer with a ruler grid, which pointer is selectively slidable relative to said block member and at right angles to said vertical pole.

10. A method of measuring the spacial orthogonal relationships between selected bony points on a subject, comprising the steps of:
(a) providing an anthropometric orthogonal frame system comprising a base and a foot frame device and an orthogonal frame device selectively positioned on said base, said orthogonal frame device comprising means mounted to said base for horizontally supporting a crossbar in selected vertical positions, said crossbar having means for supporting a first measurement device for lateral measurements, said crossbar including means for supporting a second measurement device for vertical measurements and said means for supporting said second measurement device also supporting a third measurement device for front to back measurements, the third measurement device including a pointer and said foot frame device configured to permit placing of feet of the subject in a preferred or selected stance orientation on said base;
(b) locating the feet of the subject on the base in association with said foot frame device;
(c) locating said crossbar at a selected height relative to the area of the subject to be measured;
(d) locating said pointer associated with said third measurement device at a desired location on the subject and recording measurements from at least two of said first, second and third measurement devices;
(e) locating said pointer associated with said third measurement device at a second desired location on the subject who remains stationary in the same position as when said first measurements were recorded; and
(f) recording said second measurement; and
(g) determining the spacial relationship between said first and second locations from the measurements recorded.

11. The method of claim 10 wherein prior to step (f) said foot frame device is rotated 180 on said base, said method further comprising:
(h) locating the feet of said subject in association with said rotated foot frame device, so the subject is rotated 180;
(i) repeating the steps (c) to (f) for other selected locations on another other side of the subject's body as rotated;
(j) determining the spacial relationship between the locations on one side of the subject's body with those on the other side of the body.
